(19) United States
(12) Patent Application Publication
(10) Pub. No.: US 2004/0121969 A1
(43) Pub. Date: Jun. 24, 2004

(54) Antiviral nucleoside derivatives

(75) Inventors: Robert Than Hendricks, San Carlos, CA (US); Eric Roy Humphreys, San Bruno, CA (US); Joseph Armstrong Martin, Menlo Park, CA (US); Anthony Prince, Los Altos, CA (US); Keshab Sarma, Sunnyvale, CA (US)

Correspondence Address: ROCHE PALO ALTO LLC, PATENT LAW DEPT. M/S A2-250, 3431 HILLVIEW AVENUE, PALO ALTO, CA, 94304, US

(73) Assignee: Roche Palo Alto LLC

(21) Appl. No.: 10/731,581

(22) Filed: Dec. 9, 2003

Related U.S. Application Data
(60) Provisional application No. 60/432,108, filed on Dec. 10, 2002.

(51) Int. Cl. A61K 31/7056; A61K 31/724; C07H 19/056

(52) U.S. Cl. 514/43; 514/58; 536/28.7

(57) ABSTRACT

The present invention relates to nucleosides of formula I wherein R¹, R² and R³ are as defined herein that modulateTh1 and Th2 immune activity, methods of using compounds according to formula I, alone or in combination therapy, for treatment of a bacterial or a viral infection, a parasite infestation, a cancer or tumor or an autoimmune disease and compositions containing prodrugs of the nucleoside of formula Ia.

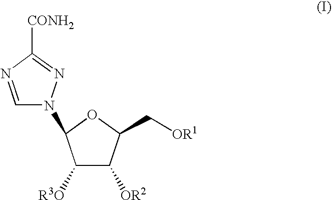

### CROSS REFERENCE TO PRIOR APPLICATION

This application claims benefit under Title 35 U.S.C. 119(e) of U.S. Provisional Application No. 60/432,108, filed Dec. 10, 2002, which is hereby incorporated by reference in its entirety.

### FIELD OF THE INVENTION

The invention relates to compounds according to formula I wherein R¹, R² and R³ are as defined herein, that modulateTh1 and Th2 immune activity. The prodrugs are easily formulated and show enhanced oral bioavailability. The compounds are prodrugs of compound Ia useful, in monotherapy or in combination therapy, for treatment of bacterial or viral infection, a parasite infestation, a cancer or tumor or an autoimmune disease. The present invention also related to compositions containing nucleosides of formula I.

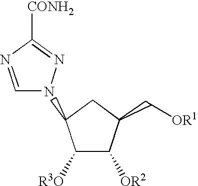

The present invention relates to prodrugs of the nucleoside of formula Ia that modulateTh1 and Th2 immune activity, methods of using prodrugs of the nuceloside of formula Ia, alone or in combination therapy, for treatment of bacterial or viral infection, a parasite infestation, a cancer or tumor or an autoimmune disease and compositions containing prodrugs of the nucleoside of formula Ia.

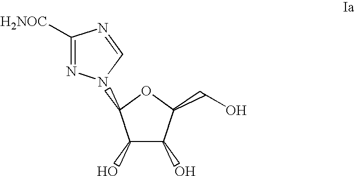

### BACKGROUND

Mammalian immune systems contain two major classes of lymphocytes: B lymphocytes (B cells), which originate in the bone marrow and are primarily responsible for humoral immunity (i.e., antibody production), and T lymphocytes (T cells) that originate in the thymus. B cells and are primarily responsible for cell-mediated immunity.

T cells can be subdivided into two subclasses, helper T cells and cytotoxic T cells. Helper T cells secrete soluble protein mediators called cytokines that activate other lymphocytes, including B cells, cytotoxic T cells, and macrophages. As used herein, lymphokines are a subset of cytokines. Type I helper T cells produce interleukin 2 (IL-2), tumor necrosis factor (TNFα) and interferon gamma (IFNΓ), and are responsible primarily for cell-mediated immunity such as delayed type hypersensitivity and antiviral immunity. Type 2 cells produce interleukins, IL4, IL5, IL-6, IL-9, IL-10 and IL-13, and are primarily involved in assisting humoral immune responses such as those seen in response to allergens, e.g. IgE and IgG4 antibody isotype switching (Mosmann, Ann. Rev. Immunol. 89 7:45-173).

These two groups of cytokines play a role in maintaining a balance between mounting an adequate immunostimulatory response required to fend off a pathogen infection, parasitic infestation and deleterious immune hyperactivity which can initiate or exacerbate immunopathological response.

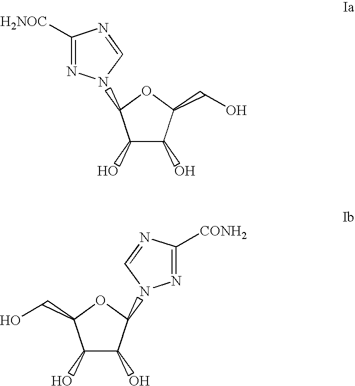

The antiviral activity exhibited by ribavirin (Ib) has been shown arise from a dual mechanism of action: direct inhibition of viral replication and enhancement of the Th1 profile. Levovirin, the enantiomer of (Ib) also exhibits a Th1-selective immunostimulatory response; however, it lacks a direct inhibition of viral replication. Thus treatment of human mononuclear cells with (Ib) resulted in elevated levels of IL-2, TNFα and IFNΓ (K. S. Ramasamy et al, J. Med. Chem 2000 46:1019-1028; M. Assenmacher et al. Eur. J. Immunol. 1998 28:1534-1543; K. Ramasamy et al., U.S. Pat. No. 6,130,326). The enhancement of cell mediated immunity can either alone, or in combination therapy, affor a useful treatment modality against a variety of pathological conditions. Compounds which demonstrate Th1 stimulatory activity afford a treatment modality for a wide variety of conditions, and in fact any condition which responds positively to administration of one or more of the compounds. Specifically contemplated applications include treatment of bacterial or viral infection, a parasite infestation, a cancer or tumor or an autoimmune disease.

Infections contemplated to be treated with the compounds of the present invention include respiratory syncytial virus (RSV), hepatitis B virus (HBV), hepatitis C virus (HCV), herpes simplex type I and 2, herpes genitalis, herpes keratitis, herpes encephalitis, herpes zoster, human immunodeficiency virus (HIV), influenza A virus, hantann virus (hemorrhagic fever), human papilloma virus (HPV), measles and fungus. Infestations contemplated to be treated with the compounds of the present invention include protozoan infestations, as well as helminth and other parasitic infestations.

Cancers or tumors contemplated to be treated include those caused by a virus, and the effect may involve inhibiting the transformation of virus-infected cells to a neoplastic state, inhibiting the spread of viruses from transformed cells to other normal cells and/or arresting the growth of virus-transformed cells.

Autoimmune and other diseases contemplated to be treated include arthritis, psoriasis, bowel disease, juvenile diabetes, lupus, multiple sclerosis, gout and gouty arthritis), rheumatoid arthritis, rejection of transplantation, allergy and asthma.

In yet another aspect, a method of treating a mammal comprises administering a therapeutically and/or prophylactically effective amount of a compound according to formula Ia where R¹, R² and R³ are as defined herein. Compounds of the present invention may modulate some portion of the mammal's immune system, especially of lymphokine Th1 and Th2 profiles. This modulation may include stimulation of both Th1 and Th2, suppression of both Th1 and Th2, stimulation of either Th1 or Th2 and suppression of the other, or a bimodal modulation in which one effect on Th1/Th2 levels (such as generalized suppression) occurs at a low concentration, while another effect (such as stimulation of either Th1 or Th2 and suppression of the other) occurs at a higher concentration.

While nucleoside derivatives such as (Ia) or (Ib) frequently possess high levels of biological activity, their therapeutic utility is often hampered by suboptimal physical properties and poor pharmacokinetics and bioavailablility that limit the amount of the nucleoside that is absorbed. Only about 15% of the dose of levovirin is absorbed systemically after oral administration. There exists a need for therapeutic agents with improved bioavailability. The availability of nucleosides derivatives with enhanced bioavailability by the oral route would be particularly advantageous.

Prodrugs, bioreversible chemical derivatives of poorly absorbed compounds, are one approach to optimizing physical properties to improve drug delivery. (W. N. Chapman and C. J. H. Porter, Adv. Drug Deliv. 1996 19:149-169; D. Fleisher et al. Adv. Drug Deliv. 1996 19:115-130) In one approach to prodrug design, chemical derivatives are prepared to optimize oil/water partition coefficients or other physical properties to enhance passive transport across mucosal membranes. Derivatives are chosen which are substrates for nonspecific enzymes present in the cytoplasm, blood, or serum and capable of cleaving the modifying group and reverting to the bioactive parent molecule after the compound is absorbed. An ideal oral prodrug should be stable to gastric fluid and intestinal chyme, be efficiently transported across intestinal membranes and be rapidly converted to the parent drug after absorption from the gastrointestinal tract. Thus “pronucleotides” can potentially circumvent problems such as activity, bioavailability or stability of the parent nucleotide.

An alternative approach exploits nonspecific active transport systems to move the prodrug across a membrane. The prodrug portion of the molecule is designed to confer recognition by the active transport system and is cleaved after transport is complete. The nonspecific peptide transporters PepT1 and PepT2 have been suggested to be useful for improving the bioavailability of poorly absorbed drugs. (P. Balimane et al. Biochem. Biophys. Res. Commun. 1998 250: 246-251; K. Sawada et al J. Pharmacol Exp. Ther. 1999 291(2):705-709; I. Rubio-Aliaga and H. Daniel, Trends Pharmacol. Sci. 2002 23(9):434-40).

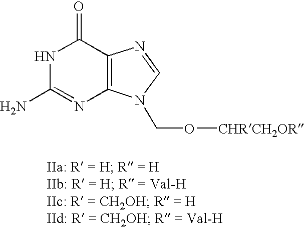

Valine esters IIb of acyclovir (Valacyclovir) IIa exhibit improved absorption characteristics which have been suggested to be the result of uptake via peptide transporters. (Balimane, supra; M. E. Ganapathy et. al. Biochem. Biophys. Res. Commun. 1998 246:470-75; P. J. Sinko and P. V. Balimane, Biopharm. Drug Dispos. 1998 19:209-17; R. L. de Vrueh et al. J. Pharmacol. Exp. Ther. 1998 286:1166-70) Mitsuru Sugara et al. (J. Pharm. Sci, 2000 89(6):781-89) suggested the improved transport of valganciclovir IId, the valine ester of ganciclovir IIc could be attributed to the PepT1 and PepT2 transport systems. WO 01/68034 A2 (G. Wang et al.) disclose bioreversible modifications of the sugar and triazole moiety of levovirin to increase drug bioavailability and to treat an infection, an infestation, a neoplasm or an autoimmune disease. WO 00/23454 (A. K. Ganguly et al.) disclose ribavirin derivatives for coadministration with interferon alfa to patients having chronic hepatitis C infection

While the availability of efficiently absorbed prodrugs affords a route to improve the bioavailabilty of levovirin, exploitation of these compounds also requires a levovirin derivative with physical properties that allow for efficient manufacture and formulation of the active ingredient. Levovirin prodrugs should possess adequate thermal stability, photostability and be non-hygroscopic. Properties relevant to the formulation chemist include particle size, polymorphic form, crystal habit, and salt form. These properties influence the aqueous solubility, dissolution profile, compatibility with other components in the formulation, route of administration and the biopharmaceutical properties. The ideal nucleoside drug candidate must then possess the physical properties which allow it to be efficiently manufactured and formulated, the pharmaceutical properties which allow it to delivered to the absorption site and chemical properties which allow recognition and uptake by the transport system and conversion back into the desired parent compound after uptake is completed.

### DETAILED DISCUSSION OF THE INVENTION

Surprisingly it has now been found that several hydrophobic amino acid ester hydrochlorides and a series of neutral mono-, di-, and triacyl derivatives of levovirin possess the requisite physical and chemical properties and exhibit improved bioavailability.

The present invention relates to nucleoside compounds according to formula Ia wherein (i) R¹, R² and R³ are independently selected from the group consisting of hydrogen, C₁₋₁₀acyl, C₁₋₁₀alkoxycarbonyl; or, (ii) R¹ is COR⁴ where COR⁴ is the hydrochloride salt of an amino acid or a dipeptide and R² and R³ are independently hydrogen, C₁₋₁₀acyl, or C₁₋₁₀alkoxycarbonyl; and, hydrates, solvates, clathrates thereof with the proviso that at least one or R¹, R² and R³ is other than hydrogen.

An embodiment of the present invention is a nucleoside compound according to formula I wherein R¹, R² and R³ are as defined hereinabove.

In another embodiment of the present invention there is provided a compound according to formula Ia wherein one of R¹ is COR⁴ and R⁴ is CH(R⁵)NH₃⁺ Cl⁻ or pyrrolidin-2-yl, R⁵ is selected from the group consisting of CH(CH₃)₂ and CH(CH₃)CH₂CH₃, and both R² and R³ are hydrogen.

In another embodiment of the present invention there is provided a compound according to formula Ia wherein one of R¹ is COR⁴, R⁴ is CH(R⁵)NH₃⁺ Cl⁻, R⁵ is CH₃, and both R² and R³ are hydrogen.

In another embodiment of the present invention there is provided a compound according to formula Ia wherein R¹, R² and R³ are independently C₁₋₁₀acyl or C₁₋₁₀alkoxycarbonyl.

In another embodiment of the present invention there is provided a compound according to formula Ia wherein the compound is: propionic acid 3S,4S-bis-propionyloxy-5S-(3-carbamoyl-[1,2,4]triazol-1-yl)-tetrahydro-furan-2S-ylmethyl ester.

In another embodiment of the present invention there is provided a compound according to formula Ia wherein R¹ is C₁₋₁₀acyl or C₁₋₁₀alkoxycarbonyl and both R² and R³ are hydrogen.

In another embodiment of the present invention there is provided a compound according to formula Ia wherein R¹ is hydrogen and both R² and R³ independently are C₁₋₁₀acyl or C₁₋₁₀alkoxycarbonyl.

In another embodiment of the present invention there is provided a compound according to formula Ia wherein the compound is: isobutyric acid 2S-(3-carbamoyl-[1,2,4]triazol-1-yl)-5S-hydroxymethyl-4S-isobutyryloxy-tetrahydro-furan-3S-yl ester; or, 2,2-dimethylpropionic acid 4S-(2,2-dimethylpropionyloxy)-5S-(3-carbamoyl-[1,2,4]triazol-1-yl)-2S-hydroxymethyl-tetrahydro-furan-3S-yl ester

In another embodiment of the present invention there is provided a method for modulating Th1 and Th2 immune activity comprising administering to a mammal a therapeutically effective amount of a compound according formula Ia wherein R¹, R² and R³ are as defined hereinabove.

In another embodiment of the present invention there is provided a method for modulating Th1 and Th2 immune activity comprising administering to a mammal a therapeutically effective amount of a compound according formula Ia wherein R¹ is COR⁴, and R⁴ is CH(R⁵)NH₃⁺ Cl⁻, R⁵ is CH(CH₃)₂ or CH(CH₃)CH₂CH₃, and both R² and R³ are hydrogen.

In another embodiment there is provided a method for treating a disease mediated by HCV comprising administering to a mammal a therapeutically effective amount of a compound according to formula I wherein one of R¹, R² and R³ is COR⁴, R⁴ is CH(R⁵)NH₃⁺ Cl⁻ or pyrrolidin-2-yl, R⁵ is CH(CH₃)₂ or CH(CH₃)CH₂CH₃, and both R² and R³ are hydrogen.

In another embodiment there is provided a method for treating a disease mediated by HCV comprising administering to a mammal a therapeutically effective amount of a compound according to formula I wherein one of R¹, R² and R³ is COR⁴, R⁴ is CH(R⁵)NH₃⁺ Cl⁻, R⁵ is CH₃ and both R² and R³ are hydrogen.

In another embodiment there is provided a method for treating a disease mediated by HCV comprising administering to a mammal a therapeutically effective amount of a compound according to formula I wherein R¹, R² and R³ are independently hydrogen C₁₋₁₀acyl or C₁₋₁₀alkoxycarbonyl.

In another embodiment there is provided a method for modulating Th1 and Th2 immune activity comprising administering to a mammal a therapeutically effective amount of a compound according to formula Ia wherein R¹, R² and R³ are as defined hereinabove in a dose of between 0.1 and 300 mg/kg of body weight of the patient/day.

In another embodiment there is provided a method for modulating Th1 and Th2 immune activity comprising administering to a mammal a therapeutically effective amount of a compound according to formula Ia wherein R¹, R² and R³ are as defined hereinabove in a dose of between 1.0 and 100 mg/kg of body weight of the patient/day.

In another embodiment there is provided a method for modulating Th1 and Th2 immune activity comprising administering to a mammal a therapeutically effective amount of a compound according to formula Ia wherein R¹, R² and R³ are as defined hereinabove in a dose of between 1.0 and 50 mg/kg of body weight of the patient/day.

In another embodiment there is provided a method for modulating Th1 and Th2 immune activity comprising administering to a human a therapeutically effective amount of a compound according to formula Ia wherein R¹, R² and R³ are as defined hereinabove

In another embodiment there is provided a method for modulating Th1 and Th2 immune activity comprising administering to a mammal a therapeutically effective amount of a compound according to formula Ia wherein R¹, R² and R³ are as defined hereinabove further comprising at least one other immune system modulator.

In another embodiment there is provided a method for modulating Th1 and Th2 immune activity comprising administering to a mammal a therapeutically effective amount of a compound according to formula Ia wherein R¹, R² and R³ are as defined hereinabove further comprising an interferon or chemically-derivatized interferon.

In another embodiment there is provided a method for modulating Th1 and Th2 immune activity comprising administering to a mammal a therapeutically effective amount of a compound according to formula Ia wherein R¹, R² and R³ are as defined hereinabove further comprising a chemically-derivatized interferon wherein said chemically derivatized interferon is PEG-interferon-α-2a (PEGASYS®) or PEG-interferon-α-2b (PEG-INTRON™).

In another embodiment there is provided a method for modulating Th1 and Th2 immune activity comprising administering to a mammal a therapeutically effective amount of a compound according to formula Ia wherein R¹, R² and R³ are as defined hereinabove further comprising at least one other antiviral, antiparasitic or anticancer compound.

A pharmaceutical composition comprising a therapeutically effective amount of a compound according to formula Ia wherein R¹, R² and R³ are as defined hereinabove claim 1 and at least one pharmaceutically acceptable carrier and optionally containing excipients.

A pharmaceutical composition comprising a therapeutically effective amount of a compound according to formula Ia wherein R¹ is COR⁴, and R⁴ is CH(R⁵)NH₃⁺ Cl⁻ or pyrrolidin-2-yl, R⁵ is CH(CH₃)₂, CH(CH₃)CH₂CH₃, or CH₃, and both R² and R³ are hydrogen and at least one pharmaceutically acceptable carrier and optionally containing excipients.

Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined hereinabove” refers to the first definition provided in the Detailed Description of the Invention.

The term “alkyl” as used herein denotes an unbranched or branched chain hydrocarbon residue containing 1 to 12 carbon atoms. The term “lower alkyl” denotes an unbranched or branched chain hydrocarbon residue containing 1 to 6 carbon atoms. Representative lower alkyl groups include methyl, ethyl, propyl, i-propyl, n-butyl, i-butyl, t-butyl or pentyl.

The term “acyl” means an organic radical of the formula R—C(O)—, formally derived from an organic acid by the removal of the hydroxyl group; the term “C₁₋₁₂acyl” refers to a acyl group wherein R is alkyl or aryl of 1-12 carbon atoms; and, the term “lower acyl” as used herein refers to a acyl group wherein R is C₁₋₆ straight, branched or cyclic alkyl. The term “aroyl” as used herein refers to an acyl group wherein R is an aryl group.

The term “alkoxy” as used herein denotes an organic radical of the formula R—O— wherein the “alkyl” portion is as defined above such as methoxy, ethoxy, n-propyloxy, i-propyloxy, n-butyloxy, i-butyloxy, t-butyloxy, pentyloxy, hexyloxy, heptyloxy including their isomers. “Lower alkoxy” as used herein denotes an alkoxy group with a “lower alkyl” group as previously defined.

The term “alkoxycarbonyl” as used herein means an organic radical of the formula R—O—C(O)— where R—O— is an alkoxy group as defined herein.

The term “naturally occurring amino acids” as used herein means the L-isomers of the naturally occurring amino acids. The naturally occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, Γ-carboxyglutamic acid, arginine, ornithine and lysine. Unless specifically indicated, all amino acids referred to in this application are in the L-form. The term “hydrophobic amino acid” as used herein glycine, alanine, valine, leucine, isoleucine, methionine, phenylalanine, tryptophan, and proline.

Compounds of formula I which are basic can form pharmaceutically acceptable salts with inorganic acids such as hydrohalic acids (e.g. hydrochloric acid and hydrobromic acid), sulphuric acid, nitric acid and phosphoric acid, and the like, and with organic acids (e.g. with acetic acid, tartaric acid, succinic acid, fumaric acid, maleic acid, malic acid, salicylic acid, citric acid, methanesulphonic acid and p-toluenesulfonic acid, and the like).

The term “solvate” as used herein means a compound of the invention or a salt, thereof, that further includes a stoichiometric or non-stoichiometric amount of a solvent bound by non-covalent intermolecular forces. Preferred solvents are volatile, non-toxic, and/or acceptable for administration to humans in trace amounts.

The term “hydrate” as used herein means a compound of the invention or a salt thereof, that further includes a stoichiometric or non-stoichiometric amount of water bound by non-covalent intermolecular forces.

The term “clathrate” as used herein means a compound of the invention or a salt thereof in the form of a crystal lattice that contains spaces (e,g., channels) that have a guest molecule (e,g.), a solvent or water) trapped within.

The term “immunomodulator” as used herein means a therapeutic agent that assists in or is capable of modifying or regulating immune functions. An agent that causes an immunological adjustment, regulation or potentiation.

The terms “Type 1” and “Type 2” responses as used herein mean the entire range of effects resulting from induction of Type 1 and Type 2 lymphocytes, respectively. Among other things, such responses include variation in production of the corresponding cytokines through transcription, translation, secretion, and possibly other mechanisms, increased proliferation of the corresponding lymphocytes, and other effects associated with increased production of cytokines, including motility effects.

The term “interferon” as used herein means the family of proteins capable of interfering with the viral infection of cells, as well as inhibiting the proliferation of normal and transformed cells, regulating cell differentiation and modulating the immune system. The four major antigenic types of interferon (α,β,Γ and ω) are defined by the cellular source of their production. Type I interferons (interferon α, β, and ω) compete with each other for cellular binding to the type I interferon receptor and thus share at least some components of this multi-subunit cell surface receptor, while the receptor for type II interferon (interferon Γ) is a distinct entity. Both naturally-occurring and recombinant interferons may be administered in combination therapy with compounds of the invention. A consensus sequence for interferon has been described in U.S. Pat. No. 4,897,471 (Y. Stabinsky).

“Antiviral drugs” as used herein refers to compounds used therapeutically or prophylactically. Antiviral intervention can occur before or at the time of viral particle attachment to the host cell membranes, during uncoating of the viral nucleic acids, by inhibiting a cellular receptor or factor required for viral replication or by blocking specific virus-coded enzymes and proteins produced by the host cell that are essential for viral replication but not for the normal host cell metabolism. Examples of antiviral compounds include, but are not limited to, idoxuridine, adenine arabinoside, trifluorothymidine, acyclovir, famciclovir, penciclovir, valacyclovir, gancicolvir, foscarnet, ribavirin, amantidine, rimantadine, cidofovir, pleconaril, relenza and tamiflu. Antiviral drugs further can include antisense oligodeoxynucleotides or phosphorothioate oligonucleotides complementary too gene sequences in target virus.

“Anticancer drugs” as used herein refers to compounds which interfere with the growth or dissemination of tumor cells. Anticancer compounds can exert a direct selective effect on the tumor cell or act indirectly to slow metastasis. Examples on anticancer drugs include, but are not limited to, altretamine, asparaginase, bleomycin,busulfan, carboplatin, chlorambucil, cisplatin, doxorubicin, leustatin, cyclophosphamide, cytarabine, stilbesterol ethinyl estradiol, etoposide, floxuridine, fludarabine, fluorouracil, flutamide, hydroxyurea, idarubicin, ifosfamide, irinotecan, leuprolide, levamisole, lomustine, mechlorethamine, medroxyprogesterone, megestrol, melphalan, mercaptopurine, methotrexate, mitomycin, mitotane, mitoxanthrone, paclitaxel, pentastatin, pipobroman, plicamycin, prednisone, procarbazine, streptozocin, tamoxifen, teniposide, vinblastine and vincristine. Anticancer drugs also can antibodies or solublized cell receptor targeting proteins expressed on cancer cells.

“Antiparasitic drugs” as used herein refer to compounds used to eliminate parasitic infestations. Antiparasitic compounds include anthelminthics, antinematodal, anticestodal, antitrematodal and antiprotozoal compounds. Examples on antiparasitic drugs include, but are not limited to, macrolide endectins, benzimidazoles, probenzimidazoles, levamisole, pyrantel, morantel , praziquantel, clorsulon, metronidazole, pyrimethamine, trimethoprim, quinacrine, quinine, mefloquine, buquinolate, decoquinate nequinate, buparvaguone.

The term “chemically-derivatized interferon” as used herein refers to an interferon molecule covalently linked to a polymer which alters the physical and/or pharmacokinetic properties of the interferon. A non-limiting list of such polymers include polyalkylene oxide homopolymers such as polyethylene glycol (PEG) or polypropylene glycol (PPG), polyoxyethylenated polyols, copolymers thereof and block copolymers thereof, provided that the water solubility of the block copolymers is maintained. One skilled in the art will be aware of numerous approaches to linking the polymer and interferon (for example, see A. Kozlowski and J. M. Harris J. Control. Release 2001 72(1-3):217-24; C. W. Gilbert and M. Park-Cho, U.S. Pat. No. 5,951,974). A non-limiting list of chemically derivatized IFNα contemplated in the present patent include PEG-interferon-α-2a (PEGASYS®) and PEG-interferon-α-2b (PEGINTRON™).

Abbreviations

The following abbreviations are used throughout this application and they have the meaning listed below:

THF: tetrahydrofuran

DMF: N,N-dimethylformamide

CBZ: benzyloxycarbonyl

PyBOP: benzotriazol-1-yloxy tris-pyrrollidino phosphonium hexafluorophosphate

IPA: isopropyl alcohol

DMAP: 4-N,N-dimethylaminopyridine

DIPEA: N,N-diisopropylethylamine

TEA: triethylamine

DEAD: diethylazodicarboxylate

PTLC: preparative thin layer chromatography

TsOH: p-toluenesulfonic acid monohydrate

Nomenclature

In general the nomenclature used in this application is based on AUTONOM™ v4.0, a Beilstein Institute computerized system for generation of IUPAC systematic nomenclature.

Examples of Compounds of the Present Invention

The following examples and preparations are provided to enable those skilled in the art to more clearly understand and to practice the present invention. They should not be considered as limiting the scope of the invention, but merely as being illustrative and representative thereof. Compounds in Table 1 are examples of mono-, di- and triacyl derivatives of levovirin. Compounds in Table 2 exemplify N-acyl levovirin derivatives.

TABLE 1
Acylated Levovirin Derivatives

(I)

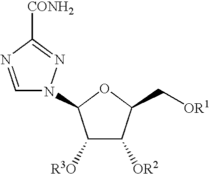

| No. | R¹ | R² | R³ | Salt | Method³ | m.s.⁴ | m.p.⁶ |
|---|---|---|---|---|---|---|---|
| 1 | MeCO | MeCO | MeCO | | A | 371 | |
| 2 | EtCO | EtCO | EtCO | | A | 413 | |
| 3 | H | Val | Val | HCl | C | 477 | 202-205 |
| 4 | Val | H | H | Tos | B | 344 | 110-114.5 |
| 5 | Val | H | H | HCl | B | 344 | 154-156 |
| 6 | (D)-Val | H | H | Tos | B | 344 | |
| 7 | Ala | H | H | Tos | B | 316 | 108-120 |
| 8 | Phe | H | H | Tos | B | 392 | 114-136 |
| 9 | Leu | H | H | Tos | B | 358 | 112-123 |
| 10 | Ile | H | H | Tos | B | 358 | 101.8-110.8 |
| 11 | t-BuCO | H | H | — | B | 329 | 139-141.6 |
| 12 | i-PrCO | H | H | — | B | 315 | 169-171.2 |
| 13 | Gly | H | H | Tos | B | 302 | 89.3-96.4 |
| 14 | MeNHCH₂CO | H | H | Tos | B | 316 | 69.4-86.3 |
| 15 | H | t-BuCO | t-BuCO | — | C | | |
| 16 | i-Pr-OCO | H | H | — | B | 329 | 46-59 |
| 17 | H | i-PrCO | i-PrCO | — | C | 407⁵ | 179.0-179.6 |
| 18 | H-Val-Pro | H | H | HCl | B | 441 | 146-149 |
| 19 | H | EtCO | EtCO | — | C | 357 | 154.2-155.6 |
| 20 | n-PrCO | n-PrCO | n-PrCO | — | A | 455 | |
| 21 | Val | EtCO | EtCO | Tos | D | 456 | 60.0-63.5 |
| 22 | Val | i-PrCO | i-PrCO | Tos | D | 506⁵ | 72.0-76.0 |
| 23 | H-Pro-Val¹ | H | H | Tos | B | 441 | 76-92 |
| 24 | EtOCO | EtOCO | EtOCO | — | A | 461 | |
| 25 | PhCO | PhCO | PhCO | — | A | 579 | |
| 26 | i-PrCO | i-PrCO | i-PrCO | — | A | 477⁵ | |
| 27 | t-BuCO | t-BuCO | t-BuCO | — | A | 519⁵ | |
| 28 | H | PhCO | PhCO | — | C | 453 | |
| 29 | t-BuCO | H | t-BuCO | — | C | 413 | |
| 30 | H | n-PrCO | n-PrCO | — | C | 407 | 135.3-135.9 |
| 31 | n-C₆H₁₃CO | H | H | — | B | 357 | 151.2-152.8 |
| 32 | n-PrOCO | n-PrOCO | n-PrOCO | — | A | 503 | 51.7-56.6 |
| 33 | H-Pro-Val² | H | H | Tos | B | 441 | 120-136 |
| 34 | C₇H₁₅CO | H | H | — | B | 371 | 154.4-155.8 |
| 35 | C₈H₁₇CO | H | H | — | B | 385 | 155-157.1 |
| 36 | EtCO | H | H | — | B | 301 | 178-181.8 |
| 47 | H | H | EtCO | — | C | 301 | |
| 48 | H | H | t-BuCO | — | C | 329 | |
| 49 | H | H | i-PrCO | — | C | 315 | |
| 51 | H | n-BuCO | n-BuCO | — | C | 435⁵ | 115.0-118.1 |
| 52 | H | n-C₅H₁₁CO | n-C₅H₁₁CO | — | C | 463⁵ | 114.8-115.3 |
| 53 | H | n-PrOCO | n-PrOCO | — | C | 417 | 101.0-103.0 |
| 54 | H | c-C₆H₁₁CO | c-C₆H₁₁CO | — | C | 487 | 195.6-197.5 |
| 55 | (n-Pr)₂CHCO | H | H | — | B | 393 | 179.0-179.9 |
| 56 | c-C₆H₁₁CO | H | H | — | B | 355 | 168.5-171.9 |
| 57 | n-C₇H₁₅OCO | H | H | — | B | 387 | 111.1-114.5 |
| 58 | H | (Et)₂CHCO | (Et)₂CHCO | — | C | 463⁵ | 154.9-160.3 |
| 59 | n-C₈H₁₇OCO | H | H | — | B | 401 | 126.3-129.1 |

Val = i-PrCHCH(NH₂)CO
Leu = Me₂CHCH₂CH(NH₂)CO
Phe = PhCH₂CH(NH₂)CO
Ile = EtCH(Me)CHNH₂CO
Ala = CH₃CH(NH₂)CO
Gly = NH₂CH₂CO
H-Val-Pro =

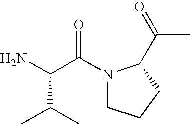

H-Pro-Val =

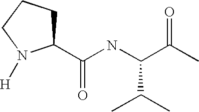

¹more polar isomer
²less polar isomer
³The methods listed herein refer to designations in the Examples (infra).
⁴mass spectra (M + H)⁺
⁵(M + Na)⁺
⁶melting point (° C.)

TABLE 2
Acylated N-Acyl Levovirin Derivatives

(II)

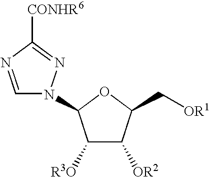

| No. | R¹ | R² | R³ | R⁶ | m.s. | Method |
|---|---|---|---|---|---|---|
| 37 | EtCO | EtCO | EtCO | EtCO | 469 | E |
| 38 | H | n-PrCO | n-PrCO | n-PrCO | 477 (M + Na)⁺ | E |
| 39 | n-PrCO | n-PrCO | n-PrCO | n-PrCO | 547 (M + Na)⁺ | E |
| 40 | H | H | H | n-PrCO | 315 | E |
| 50 | n-PrCO | H | H | n-PrCO | 477 (M +Na)⁺ | E |

Preparation of Compounds

The compounds of formula I may be prepared by various methods known in the art of organic chemistry in general and nucleoside analogue synthesis in particular. The starting materials for the syntheses are either readily available from commercial sources or are known or may themselves be prepared by techniques known in the art. The following examples (infra) are given to enable those skilled in the art to more clearly understand and to practice the present invention. They should not be considered as limiting the scope of the invention, but merely as being illustrative and representative thereof. General reviews of the preparation of nucleoside analogues are included in the following publications:

A M Michelson “The Chemistry of Nucleosides and Nucleotides”, Academic Press, New York 1963.

L Goodman “Basic Principles in Nucleic Acid Chemistry” Ed P O P Ts'O, Academic Press, New York 1974, Vol. 1, chapter 2.

“Synthetic Procedures in Nucleic acid Chemistry” Ed W W Zorbach and R S Tipson, Wiley, New York, 1973, Vol. 1 and 2.

H. Vorbrüggen and C. Ruh-Pohlenz (eds) “Handbook of Nucleoside Synthesis” Wiley, New York, 2001.

Efforts have been made to ensure accuracy with respect to numbers used (e.g., amounts, temperatures), but allowance for some experimental error and deviation, including differences in calibration, rounding of numbers, and the like, is contemplated.

Formulations and Administration

Formulations of compounds of formula I may be prepared by processes known in the formulation art. The following examples (infra) are given to enable those skilled in the art to more clearly understand and to practice the present invention. They should not be considered as limiting the scope of the invention, but merely as being illustrative and representative thereof.

While nucleoside derivatives of the present invention are optimized for delivery across the gastrointestinal mucosa, these compounds can be efficacious when administered by other routes of administration including continuous (intravenous drip) topical parenteral, intramuscular, intravenous, subcutaneous, transdermal (which may include a penetration enhancement agent), buccal, nasal and suppository administration, among other routes of administration. Oral administration can be in the form of tablets, coated tablets, dragées, hard and soft gelatine capsules, solutions, emulsions, syrups, or suspensions

For the manufacture of pharmaceutical preparations, the nucleoside derivatives, as well as their pharmaceutically useable salts, can be formulated with a therapeutically inert, inorganic or organic excipient for the production of tablets, coated tablets, dragées, hard and soft gelatine capsules, solutions, emulsions or suspensions. The compounds of formula I can be formulated in admixture with a pharmaceutically acceptable carrier. For example, the compounds of the present invention can be administered orally as pharmacologically acceptable salts. Because the compounds of the present invention are mostly water soluble, they can be administered intravenously in physiological saline solution (e.g., buffered to a pH of about 7.2 to 7.5). Conventional buffers such as phosphates, bicarbonates or citrates can be used in the present compositions. Suitable excipients for tablets, coated tablets, dragées, and hard gelatin capsules are, for example, lactose, corn starch and derivatives thereof, talc, and stearic acid or its salts. If desired, the tablets or capsules may be enteric-coated or sustained release by standard techniques. Suitable excipients for soft gelatine capsules are, for example, vegetable oils, waxes, fats, semi-solid and liquid polyols. Suitable excipients for injection solutions are, for example, water, saline, alcohols, polyols, glycerin or vegetable oils. Suitable excipients for suppositories are, for example, natural and hardened oils, waxes, fats, semi-liquid or liquid polyols. Suitable excipients for solutions and syrups for enteral use are, for example, water, polyols, saccharose, invert sugar and glucose. The pharmaceutical preparations can also contain preservatives, solubilizers, stabilizers, wetting agents, emulsifiers, sweeteners, colorants, flavorants, salts for adjustment of the osmotic pressure, buffers, masking agents or antioxidants. The pharmaceutical preparations may also contain other therapeutically active agents known in the art. Suitable pharmaceutical carriers, excipients and their formulations are described in Remington: The Science and Practice of Pharmacy 1995, edited by E. W. Martin, Mack Publishing Company, 19th edition, Easton, Pa. Representative pharmaceutical formulations containing a compound of the present invention are described in Examples 13-15.

One of ordinary skill in the formulations art will also take advantage of favorable physical and pharmacokinetic parameters of the prodrug forms, where in delivering the present compounds to targeted site within the host organism or patient to maximize the intended effect of the compound. A skilled formulation scientist may modify the formulations within the teachings of the specification to provide numerous formulations for a particular route of administration without rendering the compositions of the present invention unstable or compromising their therapeutic activity.

In particular, the modification of the present compounds to render them more soluble in water or other vehicle, for example, may be easily accomplished by minor modifications (salt formulation, esterification, etc.) which are well within the ordinary skill in the art. It is also well within the ordinary skill of the art to modify the route of administration and dosage regimen of a particular compound in order to manage the pharmacokinetics of the present compounds for maximum beneficial effect in patients.

The dosage can vary within wide limits and will, of course, be adjusted to the individual requirements in each particular case. For oral administration, a daily dosage of between about 0.01 and about 100 mg/kg body weight per day should be appropriate in monotherapy and/or in combination therapy. A preferred daily dosage is between about 0.1 and about 300 mg/kg body weight, more preferred 1 and about 100 mg/kg body weight and most preferred 1.0 and about 50 mg/kg body weight per day. A typical preparation will contain from about 5% to about 95% active compound (w/w). The daily dosage can be administered as a single dosage or in divided dosages, typically between 1 and 5 dosages per day. The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The nucleoside derivatives or the medicaments thereof may be used in monotherapy or combination therapy, i.e. the treatment may be in conjunction with the administration of one or more additional therapeutically active substance(s), for example, an immune system modulator such as an interferon, interleukin, tumor necrosis factor or colony stimulating factor or an anti-inflammatory agent and/or an antiviral agent. When the treatment is combination therapy, such administration may be concurrent or sequential with respect to that of the nucleoside derivatives. Concurrent administration, as used herein thus includes administration of the agents at the same time or at different times.

The references herein to treatment extend to prophylaxis of Hepatitis C mediated diseases as well as to the treatment of existing conditions, and that the treatment of animals includes the treatment of humans as well as other mammals. Furthermore, treatment of a Hepatitis C Virus (HCV) infection, as used herein, also includes treatment or prophylaxis of a disease or a condition associated with or mediated by Hepatitis C Virus (HCV) infection, or the clinical symptoms thereof.

### EXAMPLE 1

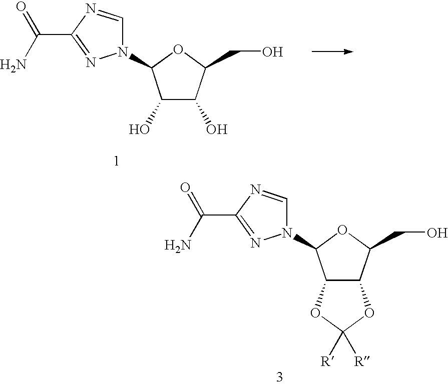

### 1-(6S-hydroxymethyl-2,2-dimethyl-tetrahydro-3aS,6aS-furo[3,4-d][1,3]dioxol-4S-yl)-1H-[1,2,4]triazole-3-carboxylic acid amide (3, R′═R″═CH₃)

Levovirin (1, 1.0 g, 4.1 mmol, Roche Carolina) was suspended in 32 mL of a 2:1 mixture of dry acetone:2,2-dimethoxypropane. The solution was stirred under N₂ in an ice bath and 7 drops of concentrated perchloric acid were added dropwise. The reaction was stirred to room temperature over 4 hours. The mixture was neutralized by addition of 1M sodium hydroxide solution and evaporated to a residue. The residue was purified via chromatography (silica gel; 5%-10% methanol/dichloromethane) to yield 0.72 g (62%)1-(6S-hydroxymethyl-2,2-dimethyl-tetrahydro-3aS,6aS-furo[3,4-d][1,3]dioxol-4S-yl)-1H-[1,2,4]triazole-3-carboxylic acid amide. (compound 41 (3, R′═R″═CH₃); (M+H)⁺=285; mp=95.1-98° C.).

### EXAMPLE 2

### 1-(6S-hydroxymethyl-2-phenyl-tetrahydro-3aS,6aS-furo[3,4-d][1,3]dioxol-4S-yl)-1H-[1,2,4]triazole-3-carboxylic acid amide (3; R′═H, R″═Ph).

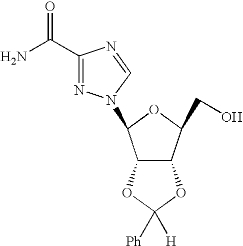

Levovirin (6.00 g, 24.5 mmol, Roche Carolina) was suspended in 60 mL of benzaldehyde. Zinc chloride (5.70 g, 41.8 mmol, Aldrich Chemical) was added to the stirred mixture. After 4 hours, the reaction mixture was added dropwise to 1 l of rapidly stirred diethyl ether. The precipitate formed was filtered, rinsed with ether and then dissolved in 350 mL of ethyl acetate and 650 mL of cold 2M sodium hydroxide solution. The layers were separated and the aqueous layer was extracted two times more with ethyl acetate. The combined ethyl acetate layers were washed with brine, dried over sodium sulfate and evaporated to a solid. The solid was triturated with ether and purified by silica gel chromatography (2%-7% methanol/dichloromethane) to yield 4.4 g (54%) 1-(6S-hydroxymethyl-2-phenyl-tetrahydro-3aS,6aS-furo[3,4-d][1,3]dioxol-4S-yl)-1H-[1,2,4]triazole-3-carboxylic acid amide (compound 42 (3, R′═Ph, R″═H); (M+H)⁺=333; mp=150-153° C.).

### EXAMPLE 3

### 1-(3R-Hydroxy-5,5,7,7-tetraisopropyl-tetrahydro-1,4,6,8-tetraoxa-5,7-disila-3aS,9aS-cyclopentacycloocten-2S-yl)-1H-[1,2,4]triazole-3-carboxylic acid amide

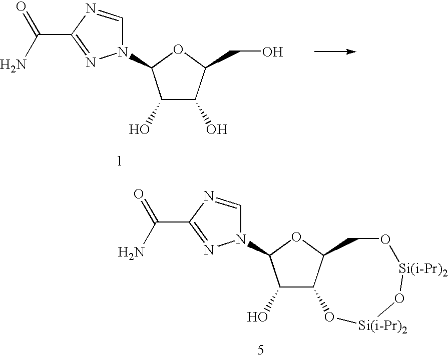

To a stirred slurry of levovirin (3.75 g, 15.4 mmol) in 30 mL of DMF were added 30 mL of pyridine, TEA (5.35 mL, 38.4 mmol), and 1,3-dichloro-1,1,3,3-tetraisopropyl-disiloxane (6.15 mL, 19.2 mmol) at 0° C. The reaction was allowed reaction to warm to room temperature and stirred for 24 hours. The resulting solution was partitioned between 1 N HCl and ethyl acetate. Organic layers were washed with brine, dried over MgSO₄ and concentrated in vacuo. The residue was purified via chromatography (25% acetone/chloroform) to yield 3.96 g (53%) 1-(3R-Hydroxy-5,5,7,7-tetraisopropyl-tetrahydro-1,4,6,8-tetraoxa-5,7-disila-3aS,9aS-cyclopentacycloocten-2S-yl)-1H-[1,2,4]triazole-3-carboxylic acid amide (compound 43).

### EXAMPLE 4

### 1-(3S,4R-dihydroxy-5S-triisopropylsilanyloxymethyl-tetrahydro-furan-2S-yl)-1H-[1,2,4]triazole-3-carboxylic acid amide

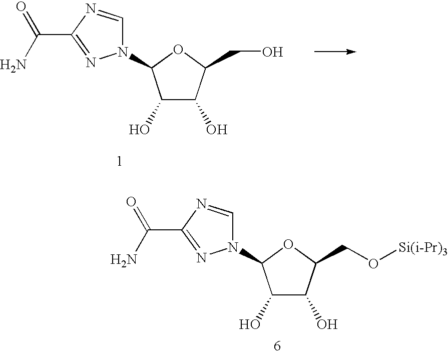

To a stirred slurry of levovirin (1, 9.22 g, 37.8 mmol) in 75 mL of DMF were added at room temperature imidazole (2.80 g, 41.1 mmol) and triisopropylsilyl chloride (8.1 mL, 38 mmol). The suspension was heated to 50° C. which dissolved the solids and temperature was maintained for 6 hrs. The solution was transferred to a separatory funnel and partitioned between 400 mL of ethyl acetate and 500 mL of water. The organic layer (slurry) was washed with 200 mL of water three times and precipitate was filtered off. Organic layer filtrate was dried over MgSO₄ and concentrated in vacuo. Filtered residue was recrystallized in 150 mL of methanol; the mother liquor was combined with organic concentrate and recrystallized further. Four crops of crystals were collected to yield 10.47 g (69%) 1-(3S,4R-dihydroxy-5S-triisopropylsilanyloxymethyl-tetrahydrofuran-2S-yl)-1H-[1,2,4]triazole-3-carboxylic acid amide as a white crystalline solid (compound 44; (M+Na)⁺=423; mp=174.6-175.7° C.).

### EXAMPLE 5

### Method A—Preparations of Trisubstituted Analogs Isobutyric acid 3S,4S-bis-isobutyrloxy-5S-(3-carbamoyl-[1,2,4]triazol-1-yl)-tetrahydro-furan-2S-ylmethyl ester (2; R¹═CH(CH₃)₂)

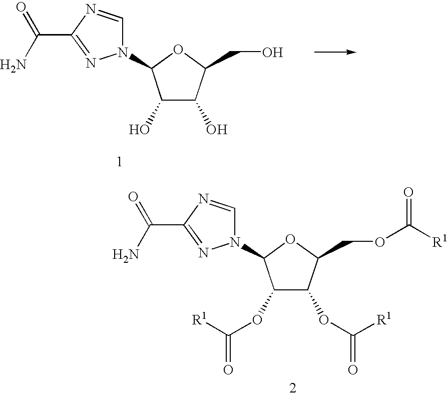

To a stirred slurry of levovirin (0.46 g, 1.88 mmol) in 8 mL of THF under nitrogen were added TEA (1.31 mL, 9.40 mmol) and isobutyric anhydride (1.41 mL, 8.48 mmol). The reaction vessel was fitted with cold finger attachment and heated to 65° C. for 24 hours. The reaction was partitioned between ethyl acetate and a saturated aqueous NaHCO₃ solution. The organic layer was washed with brine, dried over MgSO₄, and concentrated. The residue was purified via silica chromatography (3% methanol/dichloromethane) to yield 0.278 g(33%) isobutyric acid 3S,4S-bis-isobutyrloxy-5S-(3-carbamoyl-[1,2,4]triazol-1-yl)-tetrahydro-furan-2S-ylmethyl ester as a gummy solid. MS: (compound 26(2, R═CH(CH₃)₂; (M+Na)⁺=477).

Proceeding in analogous fashion with the appropriate acid anhydride there was prepared: 2,2-dimethylpropionic acid 3S,4S-bis-(2,2-dimethylpropionyloxy)-5S-(3-carbamoyl-[1,2,4]triazol-1-yl)-tetrahydro-furan-2S-ylmethyl ester (compound 27; 18%); benzoic acid 3S,4S-bis-benzoyloxy-5S-(3-carbamoyl-[1,2,4]triazol-1-yl)-tetrahydro-furan-2S-ylmethyl ester (compound 25; 66%); acetic acid 3S,4S-bis-acetoxy-5S-(3-carbamoyl-[1,2,4]triazol-1-yl)-tetrahydro-furan-2S-ylmethyl ester (compound 1; 65%; (M+H)⁺=371); propionic acid 3S,4S-bis-propionyloxy-5S-(3-carbamoyl-[1,2,4]triazol-1-yl)-tetrahydro-furan-2S-ylmethyl ester as a clear oil (compound 2; 52%; (M+H)⁺=413); butyric acid 3S,4S-bis-butyryloxy-5S-(3-carbamoyl-[1,2,4]triazol-1-yl)-tetrahydro-furan-2S-ylmethyl ester as a clear oil (compound 20; 20%; (M+H)⁺=455).

### EXAMPLE 6

### Carbonic acid 2S-(3-carbamoyl-[1,2,4]triazol-1-yl)-4S-ethoxycarbonyloxy-5S-ethoxycarbonyl-oxymethyl-tetrahydro-furan-3S-yl ester ethyl ester

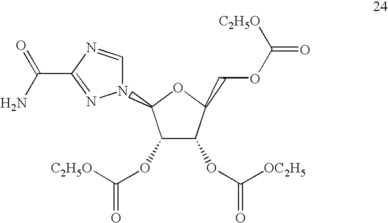

Levovirin (1, 0.5 g, 2.04 mmol, Roche Carolina) was suspended in 3 mL of DMF and 1.5 mL of pyridine. The mixture was stirred in an ice bath and ethyl chloroformate (0.78 mL, 8.19 mmol) was added slowly in three portions over 15 minutes. The reaction was stirred at room temperature for over 2 hours. Methanol was added and the reaction was stirred for 10 minutes. After evaporation, the residue was taken up in ethyl acetate and saturated ammonium chloride solution. The layers were separated and the aqueous layer was extracted with ethyl acetate once. The combined ethyl acetate layers were washed with brine and dried over sodium sulfate and concentrated. The foamy residue was purified via chromatography (3-4% methanol/dichloromethane) and lyophilization of a methanol/water solution gave solid carbonic acid 2S-(3-carbamoyl-[1,2,4]triazol-1-yl)-4S-ethoxycarbonyloxy-5S-ethoxycarbonyloxymethyl-tetrahydro-furan-3S-yl ester ethyl ester (compound 24, 74%, (M+H)⁺=461).

Proceeding in analogous fashion with the appropriate alkyl chloroformate there was prepared: carbonic acid 2S-(3-carbamoyl-[1,2,4]triazol-1-yl)-4S-propoxycarbonyloxy-5S-propoxycarbonyl-oxymethyl-tetrahydro-furan-3S-yl ester propyl ester (compound 32, 47, (M+H)⁺=503).

### Method B—Preparations of 5′-monoacyl Derivatives

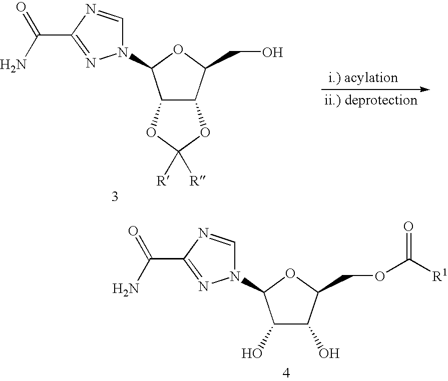

### EXAMPLE 7

### 2S-amino-3-methyl-butyric acid 5S-(3-carbamoyl-[1,2,4]triazol-1-yl)-3R,4S-dihydroxy-tetrahydro-furan-2S-ylmethyl ester; compound with toluene-4-sulfonic acid (4: R¹═CH(NH₂)CH(CH₃)₂)

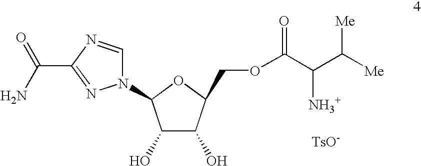

1-(6S-Hydroxymethyl-2-phenyl-tetrahydro-3aS,6aS-furo[3,4-d][1,3]dioxol-4S-yl)-1H-[1,2,4]triazole-3-carboxylic acid amide (0.49 g, 1.47 mmol) was dissolved in 5 mL of dry DMF. N-CBZ-L-valine (0.44 g, 1.77 mmol, Aldrich Chemical), PyBOP (0.84 g, 1.62 mmol, Nova Biochem) and DIPEA (0.51 mL, 2.94 mmol) were added sequentially. After stirring for 18 hr ethyl acetate and saturated ammonium chloride solution were added. The layers were separated and the aqueous layer was extracted with ethyl acetate once. The combined ethyl acetate layers were washed with water, saturated sodium bicarbonate solution, brine and dried over sodium sulfate. The solvent was evaporated and the residue was purified via chromatography (silica gel; gradient 2%-5% methanol/dichloromethane) to yield 520 mg (62%) of 2S-benzyloxycarbonylamino-3-methyl-butyric acid 6S-(3-carbamoyl-[1,2,4]triazol-1-yl)-2-phenyl-tetrahydro-3aS,6aS-furo[3,4-d][1,3]dioxol-4S-ylmethyl ester as a foam (M+H)⁺=566.

2S-Benzyloxycarbonylamino-3-methyl-butyric acid 6S-(3-carbamoyl-[1,2,4]triazol-1-yl)-2-phenyl-tetrahydro-3aS,6aS-furo[3,4-d][1,3]dioxol-4S-ylmethyl ester (0.49 g, 0.87 mmol) was dissolved in 5 mL of methanol containing 0.36 g of 20% palladium hydroxide on carbon (50 wt % water). TsOH (0.165 g, 0.87 mmol) was added and the reaction vessel was attached to a hydrogen gas-filled balloon. The vessel was purged with H₂ gas and stirred for 4.5 hr at 35° C. The mixture was then filtered through a bed of CELITE® and rinsed through with more methanol. After evaporation of solvent the residue was dissolved in water and lyophilized to give 0.44 g (98%) of 2S-amino-3-methyl-butyric acid 5S-(3-carbamoyl-[1,2,4]triazol-1-yl)-3R,4S-dihydroxy-tetrahydro-furan-2S-ylmethyl ester; compound with toluene-4-sulfonic acid. (compound 4; (M+H)⁺=344; m.p.=110-114.5° C.);

Utilizing the two step sequence described above with the appropriate carboxylic acid there was obtained: 2S-amino-propionic acid 5S-(3-carbamoyl-[1,2,4]triazol-1-yl)-3R,4S-dihydroxy-tetrahydro-furan-2S-ylmethyl ester; compound with toluene-4-sulfonic acid (compound 7; 98%; (M+H)⁺=316; m.p.=108-120° C.); 2S-amino-3-phenyl-propionic acid 5S-(3-carbamoyl-[1,2,4]triazol-1-yl)-3R,4S-dihydroxy-tetrahydro-furan-2S-ylmethyl ester; compound with toluene-4-sulfonic acid (compound 8; 91%; (M+H)⁺=392; m.p.=114-136° C.); 2S-amino-4-methyl-pentanoic acid 5S-(3-carbamoyl-[1,2,4]triazol-1-yl)-3R,4S-dihydroxy-tetrahydro-furan-2S-ylmethyl ester; compound with toluene-4-sulfonic acid (compound 9; 95%; (M+H)⁺=358; m.p.=112-123° C.); 2S-amino-3S-methyl-pentanoic acid 5S-(3-carbamoyl-[1,2,4]triazol-1-yl)-3R,4S-dihydroxy-tetrahydro-furan-2S-ylmethyl ester; compound with toluene-4-sulfonic acid (compound 10; 91%; (M+H)⁺=358; m.p.=101.8-110.8° C.); 2-methyl-propionic acid 5S-(3-carbamoyl-[1,2,4]triazol-1-yl)-3R,4S-dihydroxy-tetrahydro-furan-2S-ylmethyl ester (compound 12; 94%; (M+H)⁺=315; m.p.=169-171.2° C.); 2-amino-acetic acid 5S-(3-carbamoyl-[1,2,4]triazol-1-yl)-3R,4S-dihydroxy-tetrahydro-furan-2S-ylmethyl ester; compound with toluene-4-sulfonic acid (compound 13; 91%; (M+H)⁺=302; m.p.=89.3-96.4° C.); 2-methyl-amino-acetic acid 5S-(3-carbamoyl-[1,2,4]triazol-1-yl)-3R,4S-dihydroxy-tetrahydro-furan-2S-ylmethyl ester; compound with toluene-4-sulfonic acid (compound 14; 83%; (M+H)⁺=316; m.p.=69.4-86.3° C.).

### EXAMPLE 8

### 2S-amino-3-methyl-butyric acid 5S-(3-carbamoyl-[1.2.4]triazol-1-yl)-3R,4S-dihydroxy-tetrahydro-furan-2S-ylmethyl ester, hydrochloride (4: R¹═CH(NH₂)CH(CH₃)CH₃)

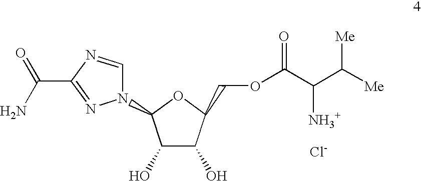

A suspension of levovirin (1.350 mg, 1.43 mmol) in 9.5 mL of THF was treated with L-Val-CBZ (360 mg, 1.43 mmol) and triphenylphosphine (600 mg, 2.29 mmol). The reaction was stirred at rt and DEAD (0.28 mL, 1.8 mmol) was added dropwise. The reaction was stirred overnight at rt and the resulting suspension was concentrated and chromatographed (PTLC, 7% MeOH/CH₂CL₂) to give 2S-benzyloxycarbonylamino-3-methyl-butyric acid 5S-(3-carbamoyl-[1,2,4]triazol-1-yl)-3R,4S-dihydroxy-tetrahydro-furan-2S-ylmethyl ester as a white solid (16%). MS: MH⁺=478 (for reference to other nucleoside Mitsunobu couplings see: Wei, Y.; Pei, D. Bioorg. Med. Chem. Lett. 2000, 10(10), 1073).

Methanol (10 mL) and 1M HCl (0.7 mL) were added to a mixture of 2S-benzyloxycarbonylamino-3-methyl-butyric acid 5S-(3-carbamoyl-[1,2,4]triazol-1-yl)-3R,4S-dihydroxy-tetrahydro-furan-2S-ylmethyl ester (170 mg, 0.35 mmol) and 50 mg of 10% Pd/carbon. The resulting suspension was stirred under a hydrogen atmosphere (about 1 atm, balloon) for 30 min and the reaction was filtered through a pad of CELITE®. The filtrate was concentrated, diluted with water, and lyophylized to give 2S-amino-3-methyl-butyric acid 5S-(3-carbamoyl-[1,2,4]triazol-1-yl)-3R,4S-dihydroxy-tetrahydro-furan-2S-ylmethyl ester, hydrochloride as a pale yellow hygroscopic solid (compound 5, 75%, (M+H)⁺=344). Recrystallization from dilute HCl/IPA gives a white crystalline solid; mp: 154-156° C.

Utilizing the two step sequence described above with the appropriate carboxylic acid there was obtained: 2R-amino-3-methyl-butyric acid 5S-(3-carbamoyl-[1,2,4]triazol-1-yl)-3R,4S-dihydroxy-tetrahydro-furan-2S-ylmethyl ester; compound with toluene-4-sulfonic acid (compound 6; 90%; (M+H)⁺=344).

### EXAMPLE 9

### 2,2-Dimethyl-propionic acid 5S-(3-carbamoyl-[1,2,4]triazol-1-yl)-3R,4S-dihydroxy-tetrahydro-furan-2S-ylmethyl ester (4: R¹═C(CH₃)₃)

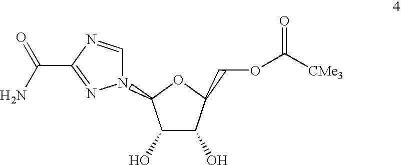

To a solution of 1-(6S-hydroxymethyl-2-phenyl-tetrahydro-3aS,6aS-furo[3,4-d][1,3]dioxol-4S-yl)-1SH-[1,2,4]triazole-3-carboxylic acid amide (0.24 g, 0.72 mmol) in 3 mL of 1:1 DMF/pyridine was added 2,2-dimethylpropionic anhydride (0.36 mL, 1.8 mmol) and DMAP (0.04 g, 0.36 mmol). The resulting solution was stirred overnight at rt and partitioned between 50 mL of ethyl acetate and sat. NH₄Cl solution. The aqueous layer was extracted with a second portion of ethyl acetate and the combined organic layers were washed with brine, dried over MgSO₄ and concentrated. The residue was chromatographed (PTLC, 5% MeOH/CH₂Cl₂) to give 2,2-dimethyl-propionic acid 6S-(3-carbamoyl-[1,2,4]triazol-1-yl)-2-phenyl-tetrahydro-3aS,6aS-furo[3,4-d][1,3]dioxol-4S-ylmethyl ester as a clear oil (95%).

Methanol (6 mL) was added to a mixture of 2,2-dimethyl-propionic acid 6S-(3-carbamoyl-[1,2,4]triazol-1-yl)-2-phenyl-tetrahydro-3aS,6aS-furo[3,4-d][1,3]dioxol-4S-ylmethyl ester (0.37 g, 0.88 mmol) and 50% wet 10% Pd(OH)₂/C (300 mg). The resulting suspension was stirred at 40° C. under a hydrogen atmosphere (about 1 atm, balloon) for 6 h and the reaction was filtered through a pad of CELITE®. The filtrate was concentrated and the resulting oil was dissolved in 1.5 mL of MeOH and 10 mL of CH₂Cl₂ and then 3 mL of hexane was added until the solution just became cloudy. The resulting precipitated white solid was filtered to give 2,2-dimethyl-propionic acid 5S-(3-carbamoyl-[1,2,4]triazol-1-yl)-3R,4S-dihydroxy-tetrahydrofuran-2S-ylmethyl ester (compound 11; 70%; (M+H)⁺=329; m.p: 139-141.6° C.);

Utilizing the two step sequence described above with the appropriate carboxylic anhydride there was obtained: heptanoic acid 5S-(3-carbamoyl-[1,2,4]triazol-1-yl)-3R,4S-dihydroxy-tetrahydro-furan-2S-ylmethyl ester (compound 31; 70%); propionic acid 5S-(3-carbamoyl-[ 1,2,4]triazol-1-yl)-3R,4S-dihydroxy-tetrahydro-furan-2S-ylmethyl ester (compound 36; 70%).

### EXAMPLE 10

### Octanoic acid 5S-(3-carbamoyl-[1,2,4]triazol-1-yl)-3R,4S-dihydroxy-tetrahydrofuran-2S-ylmethyl ester (4: R¹═C₇H₁₅)

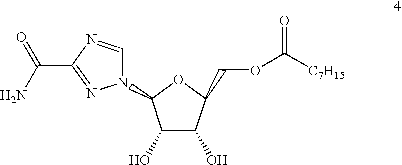

1-(6S-hydroxymethyl-2-phenyl-tetrahydro-3aS,6aS-furo[3,4-d][1,3]dioxol-4S-yl)-1H-[1,2,4]triazole-3-carboxylic acid amide (0.25 g, 0.75 mmol) was dissolved in 1 mL DMF and 0.5 mL of pyridine. The reaction solution was stirred in an ice bath and octanoyl chloride (0.16 mL, 0.94 mmol) was added dropwise. The reaction was then stirred at room temperature for 24 hr After concentration, the residue was partitioned between ethyl acetate and saturated ammonium chloride solution. The layers were separated and the aqueous layer was extracted with ethyl acetate once. The combined ethyl acetate layers were washed with brine and dried over sodium sulfate. The residue after evaporation of solvent was purified by chromatography on silica gel in 5% methanol/dichloromethane to yield 0.2 g (58%) of octanoic acid, 6S-(3-carbamoyl-[1,2,4]triazol-1-yl)-2-phenyl-tetrahydro-3aS,6aS-furo[3,4-d][1,3]dioxol-4S-ylmethyl ester was obtained; (M+H)⁺=459. Hydrogenolysis of the benzylidene group was accomplished as described in the preparation of compound 4 (supra) excluding the addition of TsOH to yield 102 mg (64%) octanoic acid 5S-(3-carbamoyl-[1,2,4]triazol-1-yl)-3R,4S-dihydroxy-tetrahydrofuran-2S-ylmethyl ester as a crystalline solid (ethyl acetate-methanol). (compound 34; (M+H)⁺=371, m.p.=154.4-155.8° C.).

Proceeding as described above with the appropriate acid chloride there was prepared: nonanoic acid 5S-(3-carbamoyl-[1,2,4]triazol-1-yl)-3R,4S-dihydroxy-tetrahydrofuran-2S-ylmethyl ester (compound 35; 82%; (M+H)⁺=385; m.p.=155-157.1);

### EXAMPLE 11

### Carbonic acid 5S-(3-carbamoyl-[1,2,4]triazol-1-yl)-3R,4S-dihydroxy-tetrahydrofuran-2S-ylmethyl ester isopropyl ester (4: R¹═O-i-C₃H₇)

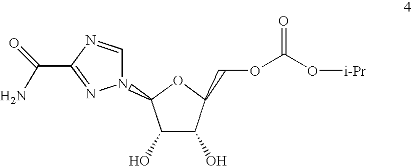

1-(6S-hydroxymethyl-2-phenyl-tetrahydro-3aS,6aS-furo[3,4-d][1,3]dioxol-4S-yl)-1H-[1,2,4]triazole-3-carboxylic acid amide (0.3 g, 0.90 mmol) was dissolved in 2.4 mL of a 1:1 mixture of dry DMF:pyridine. The reaction was placed in an ice/salt bath and stirred as iso-propylchloroformate (Aldrich 1M toluene solution) was added slowly over 20 minutes. The bath was removed and the reaction was stirred for 5 hr after which 1 mL of methanol was added and the reaction was stirred for 5 minutes more. The reaction was evaporated and the residue taken up in ethyl acetate and saturated ammonium chloride solution. The layers were separated and the aqueous layer was extracted with ethyl acetate. The combined ethyl acetate layers were washed with brine, dried over sodium sulfate and evaporated to a residue. The residue was purified by chromatography on silica gel in 5% methanol/dichloromethane to yield 150 mg (40%) carbonic acid, 6S-(3-carbamoyl-[1,2,4]triazol-1-yl)-2-phenyl-tetrahydro-3aS,6aS-furo [3,4-d][1,3]dioxol-4S-ylmethyl ester isopropyl ester (M+H)⁺=419. Deprotection of carbonic acid 6S-(3-carbamoyl-[1,2,4]triazol-1-yl)-2-phenyl-tetrahydro-3aS,6aS-furo[3,4-d][1,3]dioxol-4S-ylmethyl ester iso-propyl ester as described for compound 4, in the absence of TsOH, gave carbonic acid 5S-(3-carbamoyl-[1,2,4]triazol-1-yl)-3R,4S-dihydroxy-tetrahydrofuran-2S-ylmethyl ester isopropyl ester (compound 16; 92%; (M+H)⁺=329; m.p.=46-59° C.).

### EXAMPLE 12

### 1-(2S-Amino-3-methyl-butyryl)-pyrrolidine-2S-carboxylic acid 5S-(3-carbamoyl-[1,2,4]-triazol-1-yl)-3R,4S-dihydroxy-tetrahydro-furan-2S-ylmethyl ester, hydrochloride.(4: R1=Pro-Val-H)

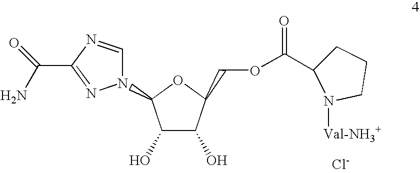

1-(6S-hydroxymethyl-2-phenyl-tetrahydro-3aS,6aS-furo[3,4-d][1,3]dioxol-4S-yl)-1H-acid amide (0.35 g, 1.05 mmol) was dissolved in 3.5 mL of dry DMF. CBZ-NH-Val-Pro-OH (0.45 g, 1.32 mmol, Bachem), PyBOP (0.68 g, 1.32 mmol, Nova Biochem) and DIPEA (0.27 mL, 1.58 mmol) were added in sequentially. After stirring for 18 hr at 35° C., ethyl acetate and saturated ammonium chloride solution were added. The layers were separated and the aqueous layer was extracted with ethyl acetate once. The combined ethyl acetate layers were washed with water, saturated sodium bicarbonate solution, brine and dried over sodium sulfate. The solvent was evaporated and the residue was purified by silica gel chromatography with 2% methanol/dichloromethane. Concentration of purified fractions yielded 370 mg (53%), 1-(2S-benzyloxycarbonylamino-3-methyl-butyryl)-pyrrolidine-2S-carboxylic acid 6S-(3-carbamoyl-[1,2,4]triazol-1-yl)-2-phenyl-tetrahydro-3aS,6aS-furo[3,4-d][1,3]dioxol-4S-ylmethyl ester was obtained as a glass (M+H)⁺=663.

Hydrogenolysis of the benzylidene protecting group was carried out as described for compound 4 (supra) replacing p-touenesulfonic acid with HCl/ether (Aldrich, 1M solution) to yield 1-(2S-amino-3-methyl-butyryl)-pyrrolidine-2S-carboxylic acid 5S-(3-carbamoyl-[1,2,4]triazol-1-yl)-3R,4S-dihydroxy-tetrahydro-furan-2S-ylmethyl ester, hydrochloride (compound 18; 79%; (M+H)⁺=441; m.p.=146-149° C.).

In analogous manner were prepared two isomers of 2-(pyrrolidine-2S-carboxamidyl)-3-methyl-butyric acid 5S-(3-carbamoyl-[1,2,4]triazol-1-yl)-3R,4S-dihydroxy-tetrahydro-furan-2S-ylmethyl ester; compound with toluene-4-sulfonic acid (compound 23, isomer 1; 88%; (M+H)⁺=441; m.p.=76−ρ° C.; compound 33, isomer 2; 92%; (M+H)⁺=441; m.p.=120-136° C.).

### EXAMPLE 13

### Method C—Preparations of Diacyl Derivatives

### Butyric acid 4S-butyryloxy-5S-(3-carbamoyl-[1,2,4]triazol-1-yl)-2S-hydroxymethyl-tetrahydro-furan-3S-yl ester (7: R¹═C₃H₇)

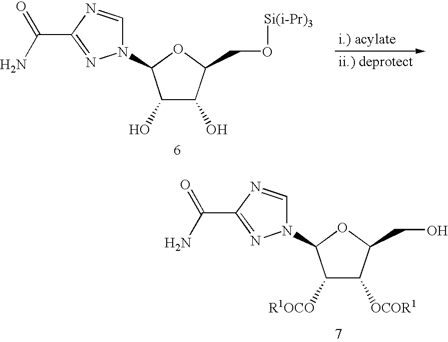

To a stirred slurry of 1-(3S,4R-dihydroxy-5S-triisopropylsilanyloxymethyl-tetrahydro-furan-2S-yl)-1H-[1,2,4]triazole-3-carboxylic acid amide (0.40 g, 1.00 mmol) in 3.3 mL of THF were added TEA (0.48 mL, 3.46 mmol), n-butyric anhydride (0.49 mL, 2.97 mmol). The reaction vessel was fitted with a cold finger attachment and heated to 65° C., under nitrogen for 17 hours. The reaction was partitioned between ethyl acetate and a saturated aqueous sodium bicarbonate solution. The organic layer was washed with brine, dried over MgSO₄, and concentrated. The residue was purified via silica gel chromatography (20% acetone/chloroform) to yield 47 g (88%) butyric acid 4S-butyryloxy-5S-(3-carbamoyl-[1,2,4]triazol-1-yl)-2S-triisopropylsilanyloxymethyl-tetrahydrofuran-3S-yl ester as a clear oil.

To a stirred solution of butyric acid 4S-butyryloxy-5S-(3-carbamoyl-[1,2,4]triazol-1-yl)-2S-triisopropylsilanyloxymethyl-tetrahydro-furan-3S-yl ester (0.47 g, 0.88 mmol) in 5 mL of acetonitrile were added 2.5 mL of 1 N H₂SO₄ at room temperature. After 16 hours, 30 mL of a saturated aqueous NaHCO₃ solution was added and product was extracted with ethyl acetate. The organic layer was washed with brine, dried over MgSO₄ and concentrated. The resulting residue was dissolved in methanol and the product precipitated with ethyl ether yielding 18 g (53%) butyric acid 4S-butyryloxy-5S-(3-carbamoyl-[1,2,4]triazol-1-yl)-2S-hydroxymethyl-tetrahydrofuran-3S-yl ester as a white crystalline solid. (compound 30; (M+Na)⁺=407; m.p.=135.3-135.9 ° C.).

Proceeding as described but using the appropriate acid anhydride there was obtained: isobutyric acid 2S-(3-carbamoyl-[1,2,4]triazol-1-yl)-5S-hydroxymethyl-4S-isobutyryloxy-tetrahydro-furan-3S-yl ester (compound 17; 38%; (M+Na)⁺=407; m.p.=179.0-179.6° C.); propionic acid 4S-propionyloxy-5S-(3-carbamoyl-[1,2,4]triazol-1-yl)-2S-hydroxymethyl-tetrahydro-furan-3S-yl ester (compound 19; 27%; (M+H)⁺=357; m.p.=154.2-155.6° C.); 2,2-dimethylpropionic acid 4S-(2,2-dimethylpropionyloxy)-5S-(3-carbamoyl-[1,2,4]triazol-1-yl)-2S-hydroxymethyl-tetrahydro-furan-3S-yl ester (compound 15; 57%);. benzoic acid 4S-benzoyloxy-5S-(3-carbamoyl-[1,2,4]triazol-1-yl)-2S-hydroxymethyl-tetrahydro-furan-3S-yl ester (compound 28; 67%).

### EXAMPLE 14

### 2S-Amino-3-methyl-butyric acid 4S-(2S-amino-3-methyl-butyryloxy)-5S-(3-carbamoyl-[1,2,4]triazol-1-yl)-2S-hydroxymethyl-tetrahydrofuran-3S-yl ester dihydrochloride (7: R¹=CH(NH₂)-i-C₃H₇)

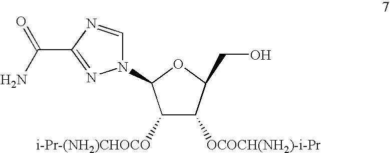

To a stirred slurry of 1-(3S,4R-dihydroxy-5S-triisopropylsilanyloxymethyl-tetrahydrofuran-2S-yl)-1H-[1,2,4]triazole-3-carboxylic acid amide (0.47 g, 1.16 mmol) in 6 mL of THF were added at room temperature 4S-isopropyl-2,5-dioxooxazolidine-3-carboxylic acid benzyl ester (0.77 g, 2.79 mmol) and 8 drops of TEA. The reaction was allowed to stir for 16 hr and was quenched with 100 mL of a saturated aqueous NaHCO₃ solution and extracted with three 100 mL portions of ethyl acetate. The organic layers were combined, washed with brine, dried over MgSO₄, and concentrated. The resulting film was purified via silica gel chromatography (15% acetone/chloroform) to yield 0.53 g (53%) 2S-benzyloxycarbonylamino-3-methyl-butyric acid 4S-(2S-benzyloxycarbonylamino-3-methyl-butyryloxy)-5S-(3-carbamoyl-[1,2,4]triazol-1-yl)-2S-triisopropylsilanyloxymethyl-tetrahydrofuran-3S-yl ester as a clear oil.

To an argon sparged, stirred solution of 2-benzyloxycarbonylamino-3-methyl-butyric acid 4-(2-benzyloxycarbonylamino-3-methyl-butyryloxy)-5-(3-carbamoyl-[1,2,4]triazol-1-yl)-2-hydroxymethyl-tetrahydro-furan-3-yl ester (0.32 g, 0.46 mmol) in 10 mL of ethanol were added hydrochloric acid (0.6 mL, 1.81 mmol) and 0.20 g of 10% Pd/C. The reaction vessel was evacuated and purged with hydrogen gas (˜1 atm) three times and left to stir for 6 hr. The suspension was filtered through CELITE® and filtrate was concentrated. The residue was dissolved in a methanol/dichloromethane (1:10) solution and the product precipitated with hexane to yield 0.09 g (38%) 2S-amino-3-methyl-butyric acid 4S-(2S-amino-3-methyl-butyryloxy)-5S-(3-carbamoyl-[1,2,4]triazol-1-yl)-2S-hydroxymethyl-tetrahydrofuran-3S-yl ester dihydrochloride as a white crystalline solid. (compound 3; (M+Cl)⁻=477; m.p.=202.0-205.0° C.).

### EXAMPLE 15

### 2,2-Dimethyl-propionic acid 2S-(3-carbamoyl-[1,2,4]triazol-1-yl)-5S-(2,2-dimethyl-propionyloxymethyl)-4S-hydroxy-tetrahydro-furan-3S-yl ester (9: R¹═C(CH₃)₃)

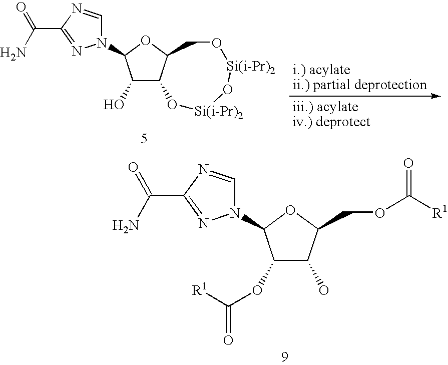

To a stirred slurry of 1-(3R-hydroxy-5,5,7,7-tetraisopropyl-tetrahydro-1,4,6,8-tetraoxa-5,7-disila-3aS,9aS-cyclopentacycloocten-2S-yl)-1H-[1,2,4]triazole-3-carboxylic acid amide (0.92 g, 1.88 mmol) in 7 mL of 1:1 DMF:pyridine were added DMAP (0.12 g, 0.94 mmol), 2,2-dimethyl propionic anhydride (0.95 mL, 4.69 mmol) and reaction was stirred for 24 hours. The reaction mixture was partitioned between ethyl acetate and a saturated aqueous ammonium chloride solution. Organic layer was washed with brine, dried over MgSO₄, and concentrated yielding 2,2-dimethyl-propionic acid 2S-(3-carbamoyl-[1,2,4]triazol-1-yl)-5,5,7,7-tetraisopropyl-tetrahydro-3aS,9aS-1,4,6,8-tetraoxa-5,7-disila-cyclopentacycloocten-3S-yl ester as a clear oil (99%).

To a stirred solution of 2,2-dimethyl-propionic acid 2S-(3-carbamoyl-[1,2,4]triazol-1-yl)-5,5,7,7-tetraisopropyl-tetrahydro-3aS,9aS-1,4,6,8-tetraoxa-5,7-disila-cyclopentacycloocten-3S-yl ester (0.48 g, 0.92 mmol) in 5 mL of acetonitrile were added 2.5 mL of I N H₂SO₄ at room temperature. After 2 hours, 30 mL of a saturated aqueous NaHCO₃ solution were added and product was extracted with ethyl acetate. The organic layer was washed with brine, dried over MgSO₄,and concentrated to give 2,2-dimethyl-propionic acid 2S-(3-carbamoyl-[1,2,4]triazol-1-yl)-4S-(3-hydroxy-1,1,3,3-tetraisopropyldisiloxanyloxy)-5S-hydroxymethyl-tetrahydro-furan-3S-yl ester (71%).

To a stirred slurry of 2,2-dimethyl-propionic acid 2S-(3-carbamoyl-[1,2,4]triazol-1-yl)-4S-(3-hydroxy-1,1,3,3-tetraisopropyldisiloxanyloxy)-5S-hydroxymethyl-tetrahydro-furan-3S-yl ester (0.38 g, 0.65 mmol) in 2.6 mL of 1:1 DMF/pyridine were added DMAP (0.40 g, 33 mmol), 2,2-dimethyl propionic anhydride (0.33 mL, 1.63 mmol) and reaction was stirred for 24 hours. The reaction was partitioned between ethyl acetate and a saturated aqueous ammonium chloride. The organic layer was washed with brine, dried over MgSO₄, and concentrated and the resulting residue was purified via chromatography (15% acetone/chloroform) yielding 2,2-dimethyl-propionic acid 2S-(3-carbamoyl-[1,2,4]triazol-1-yl)-4S-(3-hydroxy-1,1,3,3-tetraisopropyl-1,3-disiloxanyloxy)-5S-(2,2-dimethyl-propionyloxymethyl)-tetrahydro-furan-3S-yl ester (54%).

To a stirred solution of 2,2-dimethyl-propionic acid 2S-(3-carbamoyl-[1,2,4]triazol-1-yl)-4S-(3-hydroxy-1,1,3,3-tetraisopropyl-1,3-disiloxanyloxy)-5S-(2,2-dimethyl-propionyloxymethyl)-tetrahydro-furan-3S-yl ester (0.24 g, 0.35 mmol) in 5 mL of acetonitrile were added 2.5 mL of 1 N H₂SO₄ at room temperature. After 72 hours, 30 mL of a saturated aqueous NaHCO₃ solution were added and the product was extracted with ethyl acetate. The organic layer was washed with brine, dried over MgSO₄ and concentrated and the residue was purified via preparatory HPLC yielding 2,2-dimethyl-propionic acid 2S-(3-carbamoyl-[1,2,4]triazol-1-yl)-5S-(2,2-dimethyl-propionyloxymethyl)-4S-hydroxy-tetrahydro-furan-3S-yl ester (compound 29, 15%, (M+H)⁺=413).

### EXAMPLE 16

### Method D—Mixed Acyl Derivatives

### 2S-amino-3-methyl-butyric acid 5S-(3-carbamoyl-[1,2,4]triazol-1-yl)-3S,4S-bis-isobutyryloxy-tetrahydro-furan-2S-ylmethyl ester; compound with toluene-4-sulfonic acid (8: R¹=i-Pr, R²═CH(NH₂)CH(CH₃)CH₃)

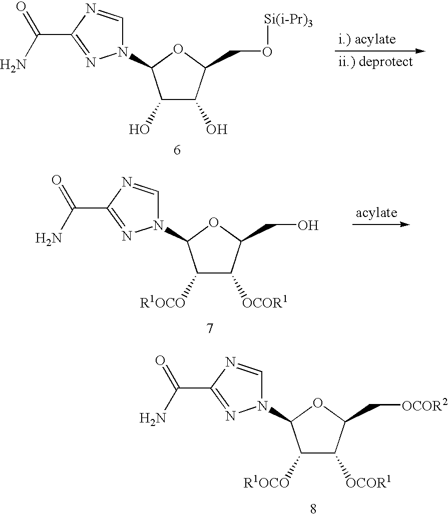

To a stirred slurry of isobutyric acid 2S-(3-carbamoyl-[1,2,4]triazol-1-yl)-5S-hydroxymethyl-4S-isobutyryloxy-tetrahydro-furan-3S-yl ester (Example 13 supra, compound 17, 0.50 g, 1.29 mmol) in 6 mL of THF were added at room temperature 4S-isopropyl-2,5-dioxo-oxazolidine-3-carboxylic acid benzyl ester (0.43 g, 1.55 mmol) and 0.3 mL of TEA. The reaction was allowed to stir for 12 hr and was quenched with 100 mL of a saturated aqueous NaHCO₃ solution and extracted with three 100 mL portions of ethyl acetate. The combined extracts were washed with brine, dried over MgSO₄, and concentrated. The residue was purified via chromatography (silica gel; 35% ethyl acetate/hexane) to yield 0.52 g (65%) 2S-benzyloxycarbonylamino-3-methyl-butyric acid 5S-(3-carbamoyl-[1,2,4]triazol-1-yl)-3S,4S-bis-isobutyryloxy-tetrahydro-furan-2S-ylmethyl ester; (M+H)⁺=484.

To an argon sparged, stirred solution of 2S-benzyloxycarbonylamino-3-methyl-butyric acid 5S-(3-carbamoyl-[1,2,4]triazol-1-yl)-3S,4S-bis-isobutyryloxy-tetrahydro-furan-2S-yl methyl ester (0.52 g, 0.84 mmol) in 10 mL of methanol were added p-toluenesulfonic acid (0.16 g, 0.84 mmol) and 0.15 g of 10% Pd/C. The reaction vessel was evacuated and purged three times with hydrogen gas (about 1 atm) and stirred for 3 hours. The slurry was then filtered through CELITE® and the resulting filtrate was concentrated, dissolved in a methanol/dichloromethane(1:10) solution and precipitated with hexane to yield 0.18 g (33%) 2S-amino-3-methyl-butyric acid 5S-(3-carbamoyl-[1,2,4]triazol-1-yl)-3S,4S-bis-isobutyryloxy-tetrahydrofuran-2S-yl methyl ester compound with toluene-4-sulfonic acid as a yellow solid (compound 22; (M+Na)⁺=506; m.p.=72.0-76.0° C.).

Proceeding as described above with propionic acid, 4S-propionyloxy-5S-(3-carbamoyl-[1,2,4]triazol-1-yl)-2S-hydroxymethyl-tetrahydro-furan-3S-yl ester gave 2S-benzyloxycarbonylamino-3-methyl-butyric acid 5S-(3-carbamoyl-[1,2,4]triazol-1-yl)-3S, 4S-bispropionyloxy-tetrahydro-furan-2S-ylmethyl ester (77%; (M+H)⁺=456) which was deprotected to yield 2S-amino-3-methyl-butyric acid 5S-(3-carbamoyl-[1,2,4]triazol-1-yl)-3S,4S-bis-propionyloxy-tetrahydro-furan-2S-ylmethyl ester; compound with toluene-4-sulfonic acid (compound 21; 46%).

### EXAMPLE 17

Caco Assay

For general discussions of the Caco Assay see: S. Yee, “In Vitro Permeability Across Caco-2 Cells (Colonic) Can Predict In Vivo (Small Intestinal) Absorption in Man—Fact or Myth” Pharm. Res. 14(6):763-766 (1997) and Yamashita et. al., “Analysis of Drug Permeation Across Caco-2 Monolayer: Implication for Predicting In Vivo Drug Absorption” Pharm. Res. 14(4):486-491(1997). For specific technical aspects see: Grass, G. M. and Sweetana, S. A. “In Vitro Measurement of Gastrointestinal Tissue Permeability Using a New Diffusion Cell” Pharm. Res. 5(6):372-376 (1988); Rubas et. al., “Comparison of the Permeability Characteristics of a Human Colonic Epithelial (Caco-2) Cell Line to Colon of Rabbit, Monkey, and Dog Intestine and Human Drug Absorption” Pharm. Res. 10(1): 113-117 (1993).

Incubation Medium and Culture Conditions:

High passage (108-120) Caco-2 cells are cultured in Dulbecco's Modified Eagle Media with high Glucose and L-Glutamine (DMEM) (Gibco/Life Technologies, Cat # 11965-084) supplemented with 10% Fetal Bovine Serum, 1× L-Glutamine (Gibco/Life Technologies, Cat # 25030-081) 1× Penicillin-streptomycin (Gibco/Life Technologies, Cat # 15140-122) 1× Non-essential Amino Acids, (Gibco/Life Technologies, Cat # 11140-019). Cells are maintained in T225 cm² Cell Culture Flask Tissue Culture Treated (Costar, Cat # 3001) at 37° C. and 5% CO₂. For transport experiments, cells are plated at 7.1×10⁴ cells/well into 12-well collagen-coated PTFE membrane polystyrene plates with inserts (Costar # 3493, 12 mm diameter, 0.4 um pore size, sterile, tissue culture treated). Cells are fed every 3 days and maintained at 37° C. and 5% CO₂ for 21 days to allow complete formation of a polarized monolayer with tight junctions.

Stock and Working Solutions:

Kreb's-Henseleit Bicarbonate Buffers pH 6.5 and 7.4

Reagents:

Distilled Water (glass distilled or Nanopure)

Kreb's-Henseleit bicarbonate buffer mix (powder, SIGMA # K-3753)

Calcium chloride dihydrate (MW=147.0)

Sodium bicarbonate (MW=84.01)

Dissolve Kreb's-Henseleit bicarbonate buffer mix in about 900 mL of water. When buffer mix is dissolved, add 0.373 gm calcium chloride dihydrate. After calcium chloride dihydrate dissolves, add 2.1 gm sodium bicarbonate, after sodium bicarbonate dissolves, add water QS to 1000 mL, then sterile filter through 0.2 μm filter and store in a refrigerator

Test/standard Compound Solutions:

Prepare 5 mg/ml stock solution of test compound in DMSO and store at 4° C. Dilute desired amount of 5 mg/ml stock to 10 mL with pH 6.5 Kreb's Henseleit bicarbonate buffer to give a concentration of 100 μM. Then 1 mL of 100 μM solution was further diluted to 5 mL to make the concentration of 20 μM. This 20 μM test solution was used as initial donor dosing solution (D0). Warm drug solution(s) to 37° C. before use.

Assay Procedure

1. Prewarm buffer, working solutions, and three 12-well plates containing buffer for each plate of 12 inserts. Using a millicell®-ERS equipped with “chopstick” electrodes (Millipore, Bedford, Mass.) check the TEER. This procedure should be done when the cells are at approximately 37° C., since TEER is effected by temperature. Use only those inserts that have TEER above 300 ohms.

2. Decant media and wash each insert once with warm Kreb's-Henseleit bicarbonate buffer.

3. 0.5 mL pH 6.5 Kreb's-Henseleit buffer was added to apical side of the cell monolayers and 1.25 mL pH 7.4 Kreb's-Henseleit buffer to the basolateral chamber. The cells were equilibrated in 37° C. and 5% CO₂ incubator for at least 30 minutes.

4. The apical side buffer was removed and replaced with 0.5 mL 20 μM test solutions.

5. The cells were then incubated at 37° C. and 5% CO₂.

6. At 30, 60 and 90 minutes time points, the inserts were transferred to new plates which receiver sides contained 1.25 mL warm fresh pH 7.4 Kreb's-Henseleit buffer.

7. The media from all plates were collected as receiver samples.

8. After 60 min transport studies, Lucifer Yellow (0.05 mL×1000 μM) was added to the apical side of the wells. At the end of the transport studies (90 minutes), the fluorescence of the receiver side samples was measured.

Sample solutions from the donor side were collected at the end of the experiments as D90 samples. The dC/dt of test substance was calculated from sampling data at 30 (assume 0 ng/mL) and 60 minutes. The apparent permeability coefficient (Pₐₚₚ) was calculated from the following equation,

$${P}_{app} = \frac{ⅆ Q}{ⅆ t} \times \frac{1}{A \times {C}_{0}} = \frac{ⅆ C}{ⅆ t} \times \frac{V}{A \times {C}_{0}}$$

where dQ in the change in amount of compound in receiver, dC is the change in the concentration of compound in receiver, V is the volume (cm³) of the receiver solution, A is the surface area (cm²) of the insert, Cₒ is the ‘initial’ concentration of drug substance, and dC/dt is the change in drug substance concentration in the receiver solution over the 90 minute incubation time, i.e., the slope (μg/cm³/sec) of the drug substance concentration in the receiver solution vs. time.

TABLE 4
Caco-2 cell assay permeability of selected comp unds

| | Caco-2 permeability |
|---|---|
| Compound number | (×10⁻⁶ cm/sec) |
| 2 | 7.8 |
| 3 | 8.6 |
| 5 | 5.6 |
| 7 | 1.2 |
| 9 | 0.9 |
| 10 | 1.5 |
| 17 | 4.8 |
| 18 | 3.4 |
| 28 | 26.6 |
| 34 | 6.5 |

### EXAMPLE 18

Formulations Composition for Oral Administration

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

Composition for Oral Administration

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

Composition for Oral Administration

| Ingredient | Amount | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | mL |
| Colorings | 0.5 | mg |
| Distilled water | q.s. to 100 | mL |

The ingredients are mixed to form a suspension for oral administration.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

We claim:

1. A compound according to formula I 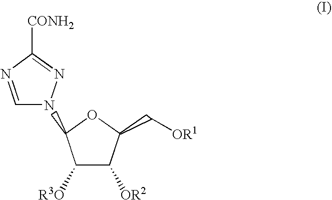
wherein (i) R¹, R² and R³ are independently selected from the group consisting of hydrogen, C₁₋₁₀acyl, C₁₋₁₀alkoxycarbonyl; or, (ii) R¹ is COR⁴ where COR⁴ is the hydrochloride salt of an amino acid or a dipeptide and R² and R³ are independently hydrogen, C₁₋₁₀acyl, or C₁₋₁₀alkoxycarbonyl; and, hydrates, solvates, clathrates thereof; with the proviso that at least one or R¹, R² and R³ is not hydrogen.

2. A compound according to claim 1 wherein R¹ is COR⁴, and R⁴ is CH(R⁵)NH₃⁺ Cl or pyrrolidin-2-yl, R⁵ is selected from the group consisting of CH(CH₃)₂ and CH(CH₃)CH₂CH₃, and both R² and R³ are hydrogen.

3. A compound according to claim 1 wherein R¹ is COR⁴, and R⁴ is CH(R⁵)NH₃⁺ Cl⁻, R⁵ is CH₃, and both R² and R³ are hydrogen.

4. A compound according to claim 1 wherein R¹, R² and R³ are independently C₁₋₁₀acyl or C₁₋₁₀alkoxycarbonyl.

5. A compound according to claim 4 wherein the compound is: propionic acid 3S,4S-bis-propionyloxy-5S-(3-carbamoyl-[1,2,4]triazol-1-yl)-tetrahydro-furan-2S-ylmethyl ester

6. A compound according to claim 1 wherein R¹ is C₁₋₁₀acyl or C₁₋₁₀alkoxycarbonyl and both R² and R³ are hydrogen.

7. A compound according to claim 1 wherein R¹ is hydrogen and both R² and R³ independently are C₁₋₁₀acyl or C₁₋₁₀alkoxycarbonyl.

8. A compound according to claim 7 wherein the compound is:
isobutyric acid 2S-(3-carbamoyl-[1,2,4]triazol-1-yl)-5S-hydroxymethyl-4S-isobutyryloxy-tetrahydro-furan-3S-yl ester; or,
2,2-dimethylpropionic acid 4S-(2,2-dimethylpropionyloxy)-5S-(3-carbamoyl-[1,2,4]triazol-1-yl)-2S-hydroxymethyl-tetrahydro-furan-3S-yl ester

9. A method for modulating Th1 and Th2 immune activity comprising administering to a mammal a therapeutically effective amount of a compound according to claim 1.

10. A method according to claim 9 wherein R¹ is COR⁴, and R⁴ is CH(R⁵)NH₃⁺ Cl⁻ or pyrrolidin-2-yl, R⁵ is CH(CH₃)₂ or CH(CH₃)CH₂CH₃, and both R² and R³ are hydrogen.

11. A method according to claim 9 wherein R¹ is COR⁴, and R⁴ is CH(R⁵)NH₃⁺ Cl, R⁵ is C1H₃, and both R² and R³ are hydrogen.

12. A method according to claim 9 wherein R¹, R² and R³ are independently hydrogen, C₁₋₁₀acyl or C₁₋₁₀alkoxycarbonyl.

13. The method of claim 9 wherein the compound is delivered in a dose of between 0.1 and 300 mg/kg of body weight of the patient/day.

14. The method of claim 9 wherein the compound is delivered in a dose of between 1.0 and 100 mg/kg of body weight of the patient/day.

15. The method of claim 9 wherein the compound is delivered in a dose of between 1.0 and 50 mg/kg of body weight of the patient/day.

16. The method of claim 9 wherein the mammal is a human.

17. The method of claim 9 further comprising at least one other immune system modulator.

18. The method of claim 17 wherein the immune system modulator is an interferon or chemically-derivatized interferon.

19. The method of claim 18 wherein the chemically derivatized interferon is PEG-interferon-α-2a (PEGASYS®) or PEG-interferon-α-2b (PEG-INTRON™)

20. The method of claim 9 further comprising a administering at least one other antiviral, antiparasitic or anticancer compound.

21. A pharmaceutical composition comprising a therapeutically effective amount of a compound according to claim 1 and at least one pharmaceutically acceptable carrier and optionally containing excipients.

22. A pharmaceutical composition according to claim 21 wherein R¹ is COR⁴, and R⁴ is CH(R⁵)NH₃⁺Cl⁻, R⁵ is CH(CH₃)₂, CH(CH₃)CH₂CH₃ or CH₃, and both R² and R³ are hydrogen.
